(19) United States
(12) Patent Application Publication
(10) Pub. No.: US 2016/0082148 A1
(43) Pub. Date: Mar. 24, 2016

(54) FILM

(71) Applicant: FUJIFILM CORPORATION, Tokyo (JP)

(72) Inventors: Koju ITO, Minamiashigara-shi (JP); Souichi KOHASHI, Minamiashigara-shi (JP); Hidekazu YAMAZAKI, Minamiashigara-shi (JP); Kenichiro HATA, Gamagori (JP); Katsura SUGAWARA, Gamagori (JP); Eiko GOTO, Gamagori (JP); Masatsugu SHIMOMURA, Sendai-shi (JP); Hiroshi YABU, Sendai-shi (JP); Takahito KAWANO, Sendai-shi (JP)

(73) Assignee: FUJIFILM CORPORATION, Tokyo (JP)

(21) Appl. No.: 14/851,467

(22) Filed: Sep. 11, 2015

(30) Foreign Application Priority Data
Sep. 24, 2014 (JP) 2014-194310

(51) Int. Cl. A61L 27/18; A61L 27/56

(52) U.S. Cl. CPC A61L 27/18; A61L 27/56; A61L 31/06

(57) ABSTRACT

Disclosed is a film which is able to suppress agglutination by being continuously adhered to an adhesion target portion in a biological body while suppressing a position shift. A film includes an adhesive and an adhesion inhibiting layer. The adhesive layer absorbs and maintains a liquid in a plurality of pores which are opened in one film surface and has a capillary force for adhering the adhesive layer to a first-cell group. The adhesive layer is formed of a biodegradable polymer. The adhesion inhibiting layer configures the other film surface, and inhibits adhesion between a second-cell group which is different from the first-cell group and the adhesive layer. In the adhesive layer, the pore is formed not to be penetrated in a thickness direction of the film, and thus the first-cell group and the second-cell group are separated into the one film surface side and the other film surface side.

### CROSS-REFERENCE TO RELATED APPLICATIONS

The present application claims priority under 35 U.S.C. §119 to Japanese Patent Application No. 2014-194310, filed on Sep. 24, 2014. The above application(s) is hereby expressly incorporated by reference, in its entirety, into the present application.

### BACKGROUND OF THE INVENTION

1. Field of the Invention

The present invention relates to a film, and in particular, relates to a film which is used by being detained in a biological body.

2. Description of the Related Art

A porous film in which a biodegradable material is an original material has been considered to be applied to various applications such as a cell culture substrate which is a scaffold material culturing a cell, a wound dressing, an agglutination preventive material, a hemostatic material, and the like. In particular, in the medical application of the wound dressing, the agglutination preventive material, and the hemostatic material, the porous film is able to be adhered, for example, to an affected area in a biological body due to a capillary force by using a fine concave and convex structure of a surface. For this reason, from a viewpoint of improving workability at the time of an operation with a low load with respect to a body of a patient, the porous film has been expected as a medical film compared to a method of adhering the porous film to the affected area by using an adhesive agent which is used in the related art or an operation such as suture. For example, in WO2004/089434A, as a porous film which is able to be used in a film-like agglutination preventive material, that is, an agglutination preventive film, a film which has a honeycomb structure by forming a plurality of unpenetrated pores on one film surface in a thickness direction, and is formed of a biodegradable material is disclosed.

In addition, an agglutination preventive material which is a sponge or knit (fabric) formed of oxidized cellulose has been known. Further, for example, in JP2009-506861A and JP2004-209228A, an agglutination preventive film having a multi-layered structure in which a material having an agglutination preventive effect such as gelatin is supported by a reinforcement material formed of a biodegradable polymer is proposed.

### SUMMARY OF THE INVENTION

However, when the sponge or the knit formed of oxidized cellulose described above is used in the biological body, the agglutination may still occur. In addition, when the film disclosed in WO2004/089434A which includes the plurality of unpenetrated pores formed on the one film surface in the thickness direction, and is formed of the biodegradable material is used as the agglutination preventive film, the adhesion of the cell to a smooth surface which is the other film surface is accelerated, and thus the agglutination may become strong through this film. In JP2009-506861A and JP2004-209228A, when the film is used by being adhered to the affected area in the biological body, the film is shifted from the affected area over time, and thus may not remain in a desired position.

Therefore, the present invention is to solve the problems described above, and an object of the present invention is to provide a film which is able to suppress agglutination by being continuously adhered to an adhesion target portion in a biological body while suppressing a position shift.

According to an aspect of the present invention, there is provided a film including: an adhesive layer which includes at least one opening defining an air gap which absorbs and maintains a liquid on one film surface and generates a capillary force for adhering the adhesive layer to a first cell group, and is formed of a biodegradable polymer; and an adhesion inhibiting layer which configures the other film surface, and inhibits adhesion between a second cell group different from the first cell group and the adhesive layer, in which in order to separate the first cell group and the second cell group into the one film surface side and the other film surface side, the air gap is formed not to be penetrated in a thickness direction of the film. Furthermore, here, the “opening” is able to be in the shape of a hole, a pore, a groove, and the like, and a single opening or a plurality of opening are able to be used. For example, a plurality of hole-like openings are able to be disposed, or a single groove-like opening which extends in a plurality of directions by using an arbitrary portion as a starting point is able to be disposed, or a groove-like opening in the shape of a volute is able to be disposed.

It is preferable that the adhesion inhibiting layer covers a surface of the adhesive layer on a side opposite to the one film surface, and thus the air gap is not penetrated in the thickness direction of the film.

It is preferable that adhesion inhibiting layer is formed of at least one of phospholipid, a polyethylene glycol derivative, a 2-methacryloyloxy ethyl phosphorylcholine polymer, polyoxy ethylene sorbitan fatty acid ester, polyoxy ethylene hardened castor oil, sodium dodecylsulfate, polyoxy ethylene polyoxy propylene glycol, sorbitan fatty acid ester, glycerin fatty acid ester, sucrose fatty acid ester, lecitin, saponin, and sterol.

It is preferable that the contact angle of water in the other film surface is less than or equal to 40°. Furthermore, here, the “contact angle” indicates an angle θ between a liquid surface and a solid surface in a position in which a free surface of a stationary liquid is in contact with a solid wall (the angle is obtained in the liquid), and is able to be calculated by a θ/2 method, a tangent method, a curve fitting method, and the like.

It is preferable that a diameter of the opening of the air gap on the one film surface is in a range of greater than or equal to 100 nm and less than or equal to 20 μm, a depth of the air gap is in a range of greater than or equal to 100 nm and less than or equal to 20 μm, and an aperture ratio in the one film surface is in a range of greater than or equal to 20% and less than or equal to 80%.

It is preferable that the adhesive layer has a honeycomb structure due to the opening of the air gap in the one film surface.

It is preferable that the adhesive layer is formed by causing dew condensation on a film formed of a solution in which a hydrophobic biodegradable polymer is dissolved in a hydrophobic solvent, and by evaporating the solvent and a water droplet generated due to the dew condensation.

According to the present invention, it is possible to suppress agglutination by continuously adhering a film to an adhesion target portion in a biological body while suppressing a position shift with respect to the adhesion target portion.

### BRIEF DESCRIPTION OF THE DRAWINGS

FIG. 1 is a plan view of a film which is a first embodiment.

FIG. 2 is a sectional view cut along line II-II of FIG. 1.

FIG. 3 is a sectional view cut along line III-III of FIG. 1.

FIG. 4 is an explanatory diagram illustrating a method of obtaining an aperture ratio.

FIG. 5 is an explanatory diagram of a manufacturing flow of the film.

FIG. 6 is an explanatory diagram of a dew condensation step.

FIG. 7 is an explanatory diagram of the dew condensation step.

FIG. 8 is a sectional view of a film which is a second embodiment.

FIG. 9 is a plan view of a film which is a third embodiment.

FIG. 10 is a sectional view cut along line X-X of FIG. 9.

FIG. 11 is an explanatory diagram of a manufacturing flow of the film.

### DESCRIPTION OF THE PREFERRED EMBODIMENTS

### First Embodiment

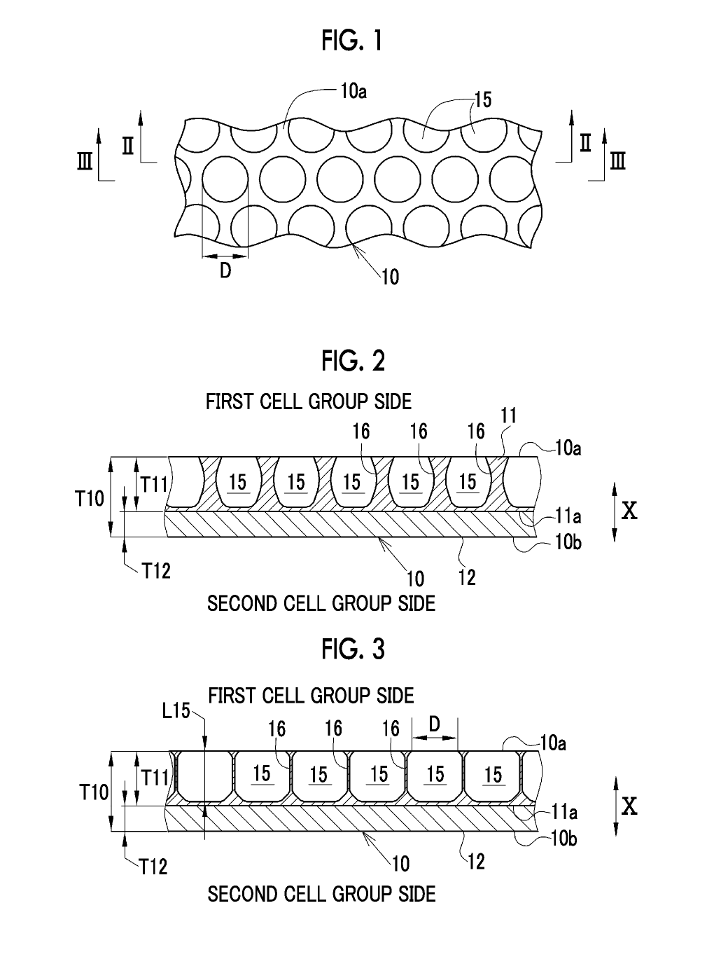

In FIG. 1 to FIG. 3, a film 10 includes an adhesive layer 11 and an adhesion inhibiting layer 12. The film 10 uses the adhesive layer 11 configuring one film surface 10a by adhering the adhesive layer 11 to a first cell group as an adhesion target portion in a biological body. The first cell group, for example, is an affected area such as a wounded portion, a suture portion sutured by an operation and the peripheral portion thereof, and the like. In the film 10 which is being adhered to the first cell group, the adhesion inhibiting layer 12 configuring the other film surface 10b is directed towards a second cell group side which is different from the first cell group.

The first cell group and the second cell group which are aggregates of a plurality of cells and are different from each other, for example, may be separated from each other in the biological body, may be separable from each other even when the first cell group is closely attached to the second cell group in a state of facing each other, or may be positioned adjacent to each other regardless of the presence or absence of a gap. Therefore, the first cell group and the second cell group may be tissues different from each other, or tissues identical to each other.

It is preferable that a thickness T10 of the film 10 is in a range of greater than or equal to 100 nm and less than or equal to 40 μm. When the thickness T10 is less than or equal to 40 μm, following properties with respect to the movement of a curved surface such as an organ are improved. In addition, when the thickness T10 is greater than or equal to 100 nm, the film 10 is easily handled.

The adhesive layer 11 is adhered to the first cell group. Furthermore, herein, “in the biological body” includes the inside of a part of a gastrointestinal system. The adhesive layer 11 has a capillary force for adhering the adhesive layer 11 to the first cell group. The adhesive layer 11 absorbs and maintains a liquid of the first cell group in a pore 15 as a plurality of air gaps which are opened in the one film surface 10a, and is adhered to the first cell group. The liquid is a liquid in the first cell group, and includes not only water but also a body fluid such as blood.

The plurality of pores 15 are arranged in the shape of a matrix along the one film surface 10a. The size and the shape of each of the pores 15 are constant, and the size of a diameter D of the opening of the pore 15 in the one film surface 10a (hereinafter, simply referred to as the diameter of the pore) and the shape thereof are also constant. When such a film 10 is observed from a direction perpendicular to the one film surface 10a, in a state where six pores 15 are arranged around each apex of a hexagon based on one arbitrary pore 15, each of the pores 15 is closely arranged. Accordingly, the film 10 has a honeycomb structure in the shape of a beehive.

In the honeycomb structure, it is not necessary that the shape of the opening of the pore 15 or the shape of a sectional surface parallel to the one film surface 10a of the pore 15 is a hexagonal shape. In this example, the shape of the opening of the pore 15 is a circular shape. The shape of the opening of the pore or the shape of the sectional surface of the pore 15, for example, may be an approximately hexagonal shape or an approximately octagonal shape which is rounded according to the density of the pore 15 per unit area of the one film surface 10a, a distance between the adjacent pores 15, and the like, and the honeycomb structure also includes such shapes. The arrangement of the pores 15 is not limited to those described above. Three to five, or seven or more pores 15 may be arranged around one arbitrary pore 15, and the pores 15 may be arranged in the shape of a square.

Each of the pores 15 is formed in the one film surface 10a as an indentation as illustrated in FIG. 2 and FIG. 3, and is not penetrated in the adhesive layer 11 in a thickness direction X. The pore 15 is not penetrated in the adhesive layer 11 in the thickness direction X, and thus the film 10 separates the first cell group on the one film surface 10a side and the second cell group on the other film surface 10b side through the film 10.

In the adhesive layer 11, a partition wall 16 is positioned between the adjacent pore 15 and pore 15 as illustrated in FIG. 2 and FIG. 3, and thus the respective pores 15 are separated. However, as described below, the partition wall 16 may not be formed, but a communication path may be formed in the film along the film surface according to the size, the density, and the like of a water droplet at the time of being formed in a casting film.

The adhesive layer 11 is formed of a biodegradable polymer, and in this embodiment, a polylactic acid (PLA) is used in the adhesive layer 11. Herein, biodegradable properties include decomposable properties in the biological body, soluble properties in the biological body, and absorbable properties in the biological body. That is, the biodegradable polymer is not limited to PLA, and may be at least one of a polymer which is decomposable in the biological body, a polymer soluble in the biological body, and a polymer absorbable in the biological body. Being decomposable in the biological body, being soluble in the biological body, and being absorbable in the biological body, for example, indicate that when the material is embedded in the body by being processed into the shape of a film or a thread, a decomposed matter is metabolized and egested in the biological body due to an operation of hydrolysis, enzyme, or the like. As the biodegradable polymer other than PLA, for example, a copolymer including PLA, polycaprolactone, a polyglycolic acid, polydioxanone, polyhydroxy butyrate, a copolymer thereof, and the like may be included. These biodegradable polymers may be independently used, or a plurality of biodegradable polymers may be used in combination.

As described below, when the adhesive layer 11 is formed by a so-called dew condensation method using dew condensation of a water droplet, from a viewpoint of forming the water droplet, it is preferable that the biodegradable polymer for the adhesive layer 11 is a hydrophobic polymer, and an amphiphilic compound which is a hydrophobic polymer and a biodegradable polymer may be used in combination. All of the respective biodegradable polymers described above are hydrophobic polymers. In addition, as the amphiphilic compound which is the biodegradable polymer, for example, phospholipid is preferable.

The adhesive layer 11 may include a component which becomes a gel by being contact with a liquid (including not only water but also a body fluid such as blood) (hereinafter, referred to as a gelling component), but it is preferable that the adhesive layer 11 does not include the gelling component. By not including the gelling component, handling properties, in particular, repasting properties in the biological body, are improved in the presence of the liquid. The repasting properties are properties of reusing the film which is pasted once, and then is peeled off or of changing a paste position by sliding the pasted film to be shifted.

The adhesive layer 11 has a porous structure in the one film surface 10a. Here, the porous structure is in a continuous structure around an air gap such as each of the pores 15 in this embodiment, and in the one film surface 10a, the air gaps are separated from each other. It is preferable that such a porous structure includes a plurality of fine air gaps of which the diameter D in the one film surface 10a is less than or equal to 20 μm. Further, it is preferable that the diameter of the air gap is approximately even. Approximately even includes both of the meaning that the diameter is even and the meaning that a variation in the diameter of the air gap decreases and a variation coefficient is less than or equal to 10%. Accordingly, in the adhesive layer 11, an adhesive force to the first cell group based on the capillary force is reliably exhibited. The variation coefficient of the diameter is obtained by using the following method. In a scanning electron microscope (SEM) photograph or an optical photomicrograph, the one film surface 10a is observed by the magnifying power such that the number of air gaps in one screen is greater than or equal to 50. On the basis of the microscopic photograph to be observed, the pores 15 in one screen are subjected to image analysis, the diameter D of each of the pores 15 is measured, an average value DAV of the diameters D and a standard deviation σD of the diameters D are obtained, and thus the variation coefficient of the diameter (unit; %) is obtained by (σD/DAV)×100.

The diameters D of the plurality of pores 15 are approximately constant in a range of approximately greater than or equal to 100 nm and less than or equal to 20 μm. In addition, a depth L15 of the pore 15 is approximately constant in a range of approximately greater than or equal to 100 nm and less than or equal to 20 μm. It is preferable that an aperture ratio SR in the one film surface 10a is in a range of greater than or equal to 20% and less than or equal to 80%. When the aperture ratio SR in the one film surface 10a is greater than or equal to 20%, the capillary force is higher than that in a case where the aperture ratio SR is less than 20%, and thus the one film surface 10a is more reliably adhered to the first cell group. In addition, when the aperture ratio SR described above is less than or equal to 80%, the surface strength of the film 10 is higher than that in a case where the aperture ratio SR is greater than 80%, and thus the porous structure such as the honeycomb structure more rarely collapses, the one film surface 10a is more reliably adhered to the first cell group, or a position shift in the affected area or the like more rarely occurs. A method of obtaining the aperture ratio SR will be described with reference to other drawings.

It is preferable that a thickness T11 of the adhesive layer 11 is in a range of greater than or equal to 100 nm and less than or equal to 20 μm. As described above, the adhesive layer 11 is formed of the biodegradable polymer, and when the thickness T11 of the adhesive layer 11 is less than or equal to 20 μm, the amount of the decomposed matter which is generated due to decomposition is suppressed to be small, and agglutination which is caused by recognizing the decomposed matter as a foreign matter in the biological body is suppressed. In addition, when the thickness T11 is greater than or equal to 100 nm, the film shape is more reliably maintained, for example, during a healing period of the affected area in the biological body even when the adhesive layer 11 is formed of the biodegradable polymer. Furthermore, within the range described above, when the thickness T11 is set according to the type of the biodegradable polymer, the affected area and the healing period thereof, and the like, a function as the film shape is maintained for a desired period in a state of suppressing the amount of the decomposed matter to be small.

The adhesion inhibiting layer 12 inhibits adhesion between the adhesive layer 11 and the second cell group. The adhesion inhibiting layer 12 is superimposed on a surface 11b of the adhesive layer 11 on a side opposite to the one film surface 10a, and is formed to cover this surface 11a. In this example, the adhesion inhibiting layer 12 is formed to cover the entire region of the surface 11a, but it is not necessary that the covered region is the entire region of the surface 11a, and for example, when the adhesion inhibiting layer 12 covers a region of approximately greater than or equal to 50% the entire region of the surface 11a, a certain effect of inhibiting the adhesion between the adhesive layer 11 and the second cell group is obtained.

The adhesion inhibiting layer 12 configures the other film surface 10b, and in this example, the other film surface 10b is flat. Here, flat indicates a state where there are no or few concave portions of which the diameter of the opening is greater than or equal to 100 nm, and specifically indicates that the aperture ratio SR in the other film surface 10b due to the concave portion of which the diameter of the opening is greater than or equal to 100 nm is less than or equal to 5%. The other film surface 10b is formed to be flat by the adhesion inhibiting layer 12, and thus the invasion of adhesive layer 11 to the cell or the like is suppressed, and when the adhesion inhibiting layer 12 is used as an agglutination preventive film, agglutination which occurs when the cell passes through the film 10 is suppressed.

It is preferable that a contact angle of water θb in the other film surface 10b is less than or equal to 40°. In the film 10, by setting the contact angle θb to be less than or equal to 40°, hydrophilic properties are higher than that in a case where the contact angle θb is greater than 40°, and thus the adhesion with respect to the second cell group is more reliably suppressed.

It is preferable that the adhesion inhibiting layer 12 contains an amphiphilic compound which includes a hydrophilic portion (for example, a hydroxy group, a carboxy group, and the like) having a hydrophilic structure, and a hydrophobic portion (for example, an alkyl group, and the like) having a hydrophobic structure. When the amphiphilic compound is used as the material of the adhesion inhibiting layer 12, the other film surface 10b may not necessarily satisfy the contact angle θb described above. As the amphiphilic compound, at least one of phospholipid, a polyethylene glycol derivative (a PEG derivative), a 2-methacryloyloxy ethyl phosphorylcholine (MPC) polymer, polyoxy ethylene sorbitan fatty acid ester, polyoxy ethylene hardened castor oil, sodium dodecylsulfate, polyoxy ethylene polyoxy propylene glycol, sorbitan fatty acid ester, glycerin fatty acid ester, sucrose fatty acid ester, lecitin, saponin, and sterol is preferable. In addition, it is preferable that a Hydrophile-Lipophile Balance (HLB) value of the amphiphilic compound to be used is in a range of greater than or equal to 1 and less than or equal to 13. When the HLB value is less than or equal to 13, the amphiphilic compound is rarely dissolved in water compared to a case where the HLB value is greater than 13 (the hydrophilic properties of the amphiphilic compound excessively increase), and thus a duration of an effect as the adhesion inhibiting layer increases. It is more preferable that the HLB value is in a range of greater than or equal to 1 and less than or equal to 8. Here, the HLB value is a value which is calculated by HLB Value=20×(weight % of a hydrophilic group) and is obtained by a Griffin method. Furthermore, the adhesion inhibiting layer 12 may be in a state where at least one of those described above is mixed in a material other than those described above, that is, a state of including at least one of those described above. By forming the adhesion inhibiting layer 12 of the materials described above, a surface of the adhesive layer 11 on a side opposite to the one film surface 10a has hydrophilic properties, and the contact angle decreases, and thus the adhesion between the adhesive layer 11 and the second cell group is more reliably suppressed.

It is preferable that a thickness T12 of the adhesion inhibiting layer 12 is in a range of greater than or equal to 100 nm and less than or equal to 20 μm. By setting the thickness T12 to be greater than or equal to 100 nm, the film shape is maintained even when the adhesive layer 11 is decomposed, and for example, a part or all of the adhesive layer 11 vanishes during the healing, and thus a healing effect is maintained for a predetermined period. In addition, by setting the thickness T12 to be less than or equal to 20 μm, the flexibility as the material is further ensured, and the following properties with respect to the movement of the surface such as an organ are more reliably obtained.

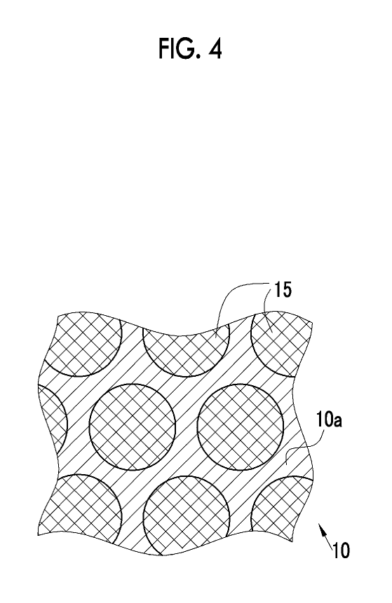

The method of obtaining the aperture ratio SR will be described with reference to FIG. 4. The aperture ratio SR is able to be calculated in each surface from which the aperture ratio SR is obtained. Here, the one film surface 10a will be described as an example of the surface from which the aperture ratio SR is obtained. When the one film surface 10a is observed from a direction perpendicular thereto, the area of the opening of the pore 15 is S15, and the area of the one film surface 10a is S10a. The area S15 of the opening of the pore 15 is the area of a portion indicated by cross-hatching in FIG. 4. The area S10a of the one film surface 10a is the area of a hatching portion indicated by oblique lines in FIG. 4. The aperture ratio SR (unit; %) in the one film surface 10a is obtained by {S15/(S10a+S15)}×100.

The operation of the configuration described above will be described. The film 10 is arranged on the first cell group side towards the adhesive layer 11, and the adhesive layer 11 is in contact with the first cell group. The liquid is included in the first cell group, each of the pores 15 absorbs and maintains the liquid of the first cell group by the capillary force, the adhesive layer 11 is adhered to the first cell group, and the adhesion is continued for a predetermined period, and thus the position shift is suppressed. The adhesive layer 11 has the honeycomb structure in which the pores 15 are closely formed, and thus the adhesion is more reliably obtained than that in a case where the pores are more sparsely formed than the pores of the honeycomb structure, and the position shift is more reliably suppressed.

Each of the pores 15 is not penetrated in the thickness direction X of the film, and thus the first cell group and the second cell group on the other film surface 10b side are separated through the film 10, and accordingly, when the first cell group is a wounded portion, the first cell group is prevented from passing through the film 10 and from advancing to the second cell group side during the healing, and thus the agglutination due to the integration of the first cell group and the second cell group is prevented. In addition, when the healing effect of the first cell group increases and the first cell group includes the suture portion sutured by the operation, for example, the healing effect is able to be obtained even when incomplete suture is performed. The adhesive layer 11 is formed of the biodegradable polymer, and thus the material of the adhesive layer 11 is decomposed or absorbed in the biological body, or is discharged according to the case.

The adhesion inhibiting layer 12 covers the surface 11a of the adhesive layer 11 which is formed of the biodegradable polymer, and thus even when the adhesive layer 11 is decomposed and the decomposed matter is generated, the decomposed matter is prevented from being recognized as the foreign matter. For this reason, the adhesion of the second cell group to the film 10 is suppressed, and thus the agglutination between the first cell group and the second cell group through the film 10 is suppressed.

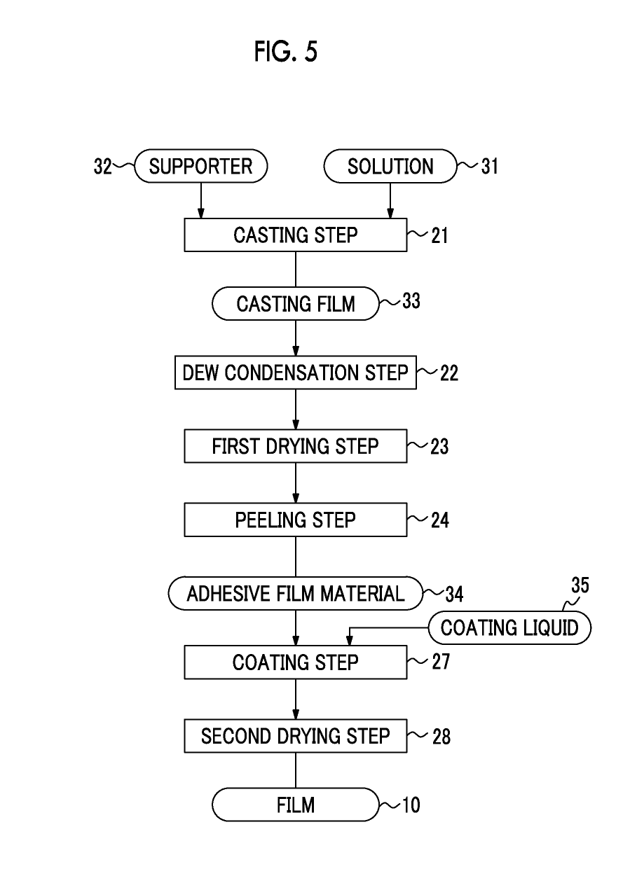

A manufacturing method of the film 10 will be described. The film 10, for example, is manufactured by a film manufacturing flow illustrated in FIG. 5. The film manufacturing flow includes a casting step 21, a dew condensation step 22, a first drying step 23, a peeling step 24, a coating step 27, and a second drying step 28. In the casting step 21, a solution 31 in which the biodegradable polymer for forming the adhesive layer 11 is dissolved in a solvent is dropped onto a supporter 32 and is spread (is casted) on the supporter 32, and thus a casting film 33 is formed. In this embodiment, as a solvent dissolving PLA which is the biodegradable polymer, dichloromethane is used. However, the solvent of PLA is not limited thereto, and chloroform and the like may be used. As a solvent of each of the biodegradable polymers described above, dichloromethane, chloroform, toluene, and the like are included. In addition, as a solvent of phospholipid having amphiphilic properties, dichloromethane, chloroform, and the like are included. As the supporter 32, in this embodiment, plate-like glass is used, but the supporter 32 is not limited thereto, and for example, a polyethylene terephthalate (PET) film and the like may be used.

In the dew condensation step 22, moisture included in the atmosphere in the vicinity of the casting film 33 is subjected to dew condensation on the film surface of the casting film 33, and thus a water droplet is formed. The details of the dew condensation step 22 will be described below. In the first drying step 23, the solvent and the water droplet formed in the dew condensation step 22 are evaporated from the casting film 33. In this first drying step 23, the solvent and the water droplet are evaporated, and thus an adhesive film material 34 for forming the adhesive layer 11 is formed on the supporter 32. Thus, the adhesive film material 34 is formed by using a dew condensation method including the dew condensation step 22 and the first drying step 23.

In the peeling step 24, the adhesive film material 34 is peeled off from the supporter 32. The peeled surface peeled off from the supporter 32 of the adhesive film material 34 is the surface 11a of the adhesive layer 11 described above. A coating liquid 35 in which a material for forming the adhesion inhibiting layer 12 is dissolved in a solvent or is dispersed in a dispersion medium is applied onto this peeled surface, and thus a coated film is formed. In the second drying step 28, the coated film is dried, and thus the adhesion inhibiting layer 12 is formed. As described above, the film 10 is manufactured. In this example, the adhesion inhibiting layer 12 is formed by coating and drying the coating liquid 35, but a method of forming the adhesion inhibiting layer 12 is not limited thereto. For example, a film material (hereinafter, referred to as an adhesion inhibit film material) which becomes the adhesion inhibiting layer 12 is prepared, and this adhesion inhibit film material is superimposed on the surface 11a of the adhesive film material 34, and thus the film 10 in which both of the adhesion inhibiting layer 12 and the adhesive film material 34 are laminated may be manufactured. When the adhesion inhibiting layer 12 includes the amphiphilic compound, it is preferable that the adhesion inhibit film material contains the amphiphilic compound at high concentration (for example, in a range of greater than or equal to 1% and less than or equal to 50%). When the mass of the adhesion inhibit film is M1 (unit: g), and the mass of the amphiphilic compound (unit: g) is M2, the concentration (unit: %) of the amphiphilic compound is a value obtained by (M2/M1)×100.

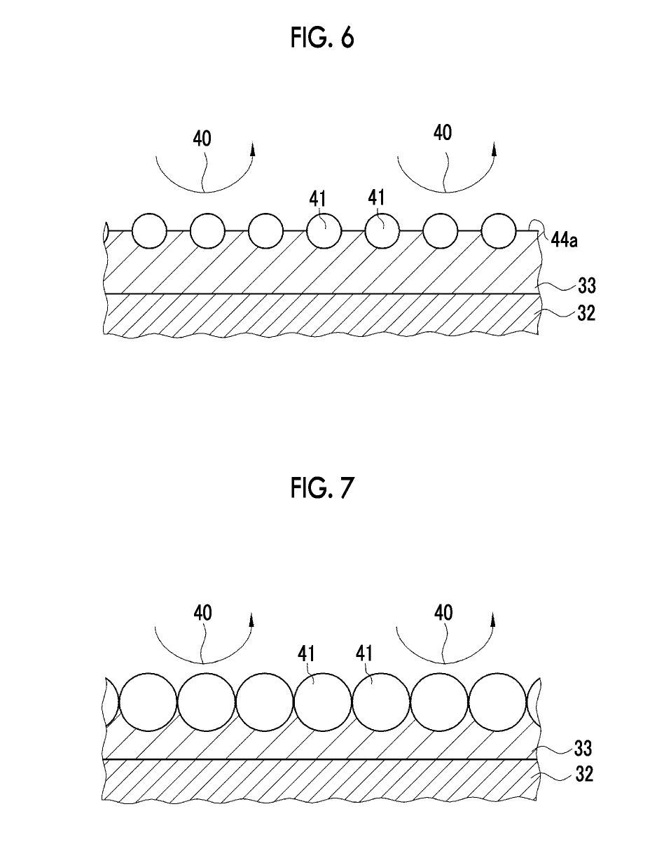

In the dew condensation step 22, as illustrated in FIG. 6, the moisture included in the atmosphere in the vicinity of the casting film 33 is subjected to the dew condensation on an exposed surface 33a of the casting film 33 on a side opposite to a surface which is in contact with the supporter 32. In this embodiment, the dew condensation is performed by supplying humidified air (hereinafter, referred to as humid air) 40 onto the exposed surface 33a, but the air may be a different gaseous body. A plurality of water droplets 41 which occur in the dew condensation grow and increase by continuing the dew condensation, and as illustrated in FIG. 7, for example, cover the exposed surface 33a without having a gap. Furthermore, the supporter 32 may be cooled, and according to this, the dew condensation is accelerated, and thus the formation and the growth of the water droplet are accelerated.

In the dew condensation step 22 described above, in order to form the adhesive layer 11 formed of the hydrophobic polymer, the polymer component of the solution 31 may be the hydrophobic polymer, and thus the water droplet 41 is formed. However, when the adhesive layer 11 formed of the hydrophilic polymer is formed, a gaseous body including a hydrophobic substance is supplied onto the exposed surface of the casting film 33 instead of the gaseous body including the moisture, and thus the liquid droplet of the hydrophobic substance may be formed on the exposed surface of the casting film. Furthermore, when the communication path along the film surface is formed in the film without including the partition wall 16, the water droplets 41 grow and are arranged such that the water droplet 41 is suitably formed by configuring a close-packed structure. For this reason, a start timing of the first drying step 23 may be set to be late such that the flux of the casting film 33 does not stop.

### Second Embodiment

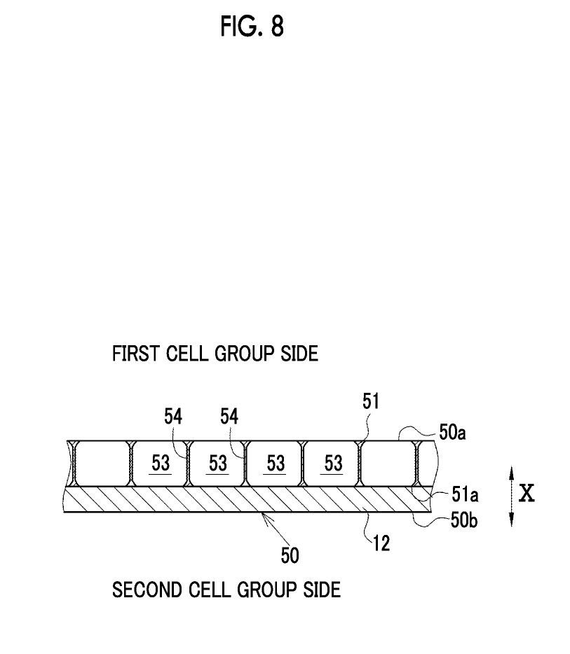

The pore 15 of the film 10 described above is not penetrated in the adhesive layer 11 in the thickness direction X, but may be penetrated. For example, as illustrated in FIG. 8, a film 50 which is a second embodiment includes an adhesive layer 51 and an adhesion inhibiting layer 12, and in the adhesive layer 51, a plurality of pores 53 are formed to be penetrated in the thickness direction X as an air gap. Furthermore, the same reference numerals are applied to the same members as those of the film of the first embodiment, and the description will be omitted. Each of the pores 53 is penetrated in the adhesive layer 51 in the thickness direction X.

The adhesion inhibiting layer 12 is formed on a surface 51a of the adhesive layer 51 on a side opposite to one film surface 50a in a state of blocking the penetrated pore 53. By this adhesion inhibiting layer 12, the first cell group and the second cell group are separated into the one film surface 50a side and the other film surface 50b side. Furthermore, in this example, the adjacent pore 53 and pore 53 are separated by a partition wall 54, but the configuration is not limited thereto. That is, a communication path along the one film surface 50a may be formed therein without including the partition wall 54.

The operation of the configuration described above will be described. The first cell group and the second cell group are separated into the one film surface 50a side and the other film surface 50b side, and thus the first cell group is prevented from passing through the film 50 and from advancing to the second cell group side. For this reason, the agglutination due to the integration of the first cell group and the second cell group is prevented.

Even in this adhesive layer 51, each of the pores 53 absorbs and maintains the liquid of the first cell group by the capillary force, the adhesive layer 51 is adhered to the first cell group, and the adhesion is continued for a predetermined period, and then the position shift of the film 50 is suppressed. The adhesive layer 51 has a honeycomb structure in which the pores 53 are closely formed, and thus the adhesion is more reliably obtained than that in a case where the pores are sparsely formed, and the position shift is more reliably suppressed. In addition, when the first cell group is a wounded portion, the healing effect of the first cell group is obtained.

The film 50 is able to be manufactured by the same manufacturing flow as that of the film 10. In order to form the adhesive layer 51 including the pore 53 penetrated in the thickness direction X, in the dew condensation step 22 described above, the water droplets 41 grow to be greater than the thickness of the adhesive layer 51 which is calculated on the basis of the solid content of the biodegradable polymer or the like included in the solution 31. A method for this includes setting the solid content of the solution 31 to be smaller than the solid content of the solution 31 in the first embodiment, and further accelerating the growth of the water droplet in the dew condensation step 22 more than that of the first embodiment.

### Third Embodiment

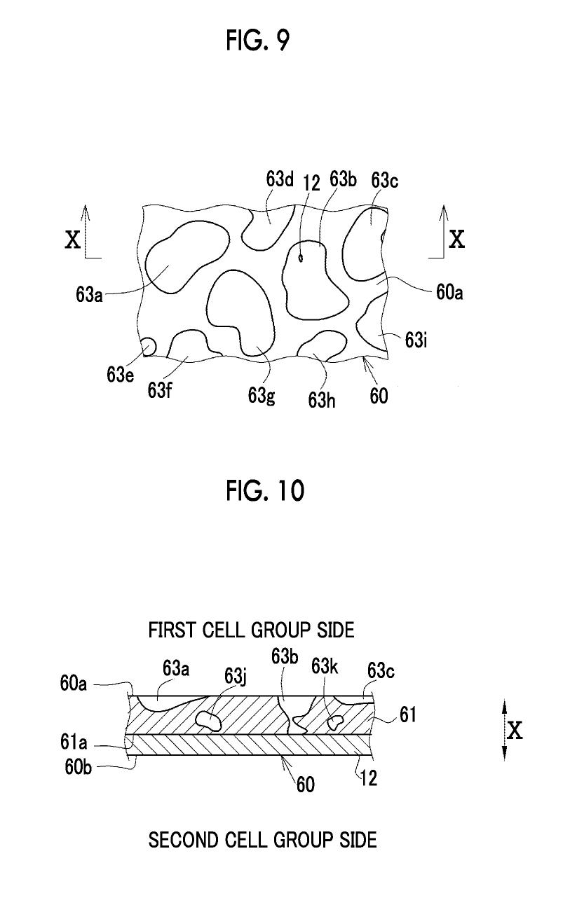

The adhesive layer is not limited to the honeycomb structure, and may be a porous structure in which the diameter of each of the openings in the one film surface as described above satisfies a range of greater than or equal to 100 nm and less than or equal to 20 μm, and the depth of the pore satisfies a range of greater than or equal to 100 nm and less than or equal to 20 μm. As illustrated in FIG. 9 and FIG. 10, a film 60 of a third embodiment includes an adhesive layer 61 and an adhesion inhibiting layer 12. In this embodiment, PLA (manufactured by Sigma-Aldrich Co. LLC., a product number of 719854) is used in the adhesive layer 61, but the configuration is not limited thereto, and as a material for forming the adhesive layer 61, the materials forming the adhesive layer 11 described above are able to be used. As with the adhesive layer 11, in the adhesive layer 61, pores 63a to 63k having different sizes and shapes are formed by being irregularly positioned as an air gap.

In these pores 63a to 63k, the pores 63a to 63i are formed to include an opening in one film surface 60a. The diameter of the opening of the pores 63a to 63i in the one film surface 50a is considered as the diameter of a circle at the time of drawing the circle having the same area as that of the opening. These pores 63a to 63i contribute to the adhesion with respect to the first cell group from the start of the contact with respect to the first cell group.

In the adhesive layer 61, the pores 63a and 63c are not penetrated in the thickness direction X, and the pore 63b is penetrated. The adhesion inhibiting layer 12 is formed in a surface 61a of the adhesive layer 61 on a side opposite to the one film surface 60a to block the penetrated pore 63b. Thus, the film 60 includes each of the pores 63a to 63i which is not penetrated in the thickness direction X such that the pores 63a to 63i are opened in the one film surface 60a. Accordingly, the first cell group and the second cell group are separated into the one film surface 50a side and the other film surface 50b side.

The pores 63j to 63k are formed in the adhesive layer 61, but are not opened in the one film surface 60a. The pores 63a to 63i contribute to the adhesion with respect to the first cell group from the start of the contact with respect to the first cell group, and the pores 63j to 63k contribute to the adhesion with respect to the first cell group from the progress of the decomposition of the biodegradable polymer configuring the adhesive layer 61. When the diameter of the opening of the pores 63j to 63k which are in contact with the first cell group due to the progress of the decomposition of the biodegradable polymer is in a range of greater than or equal to 100 nm and less than or equal to 20 μm, and the depth thereof is in a range of greater than or equal to 100 nm and less than or equal to 20 μm, the pores 63j to 63k more reliably contribute to the contact with respect to the first cell group.

The pore may be continuously formed in the one film surface 60a as a sea portion of a sea-island structure, and a certain effect is obtained in the adhesion with respect to the first cell group. However, as in this example, from a viewpoint of the adhesion with respect to the first cell group, it is preferable that the pores 63a to 63i are formed to be separated as an island portion of the sea-island structure in the one film surface 60a.

The operation of the configuration will be described. The first cell group and the second cell group are separated into the one film surface 60a side and the other film surface 60b side, and thus the first cell group is prevented from passing through the film 60 and from advancing to the second cell group side. For this reason, the agglutination due to the integration of the first cell group and the second cell group is prevented.

Even in this adhesive layer 61, each of the pores 63a to 63k absorbs and maintains the liquid of the first cell group by the capillary force, the adhesive layer 61 is adhered to the first cell group, and the adhesion is continued for a predetermined period, and then the position shift of the film 60 is suppressed. In addition, when the first cell group is a wounded portion, the healing effect of the first cell group is obtained.

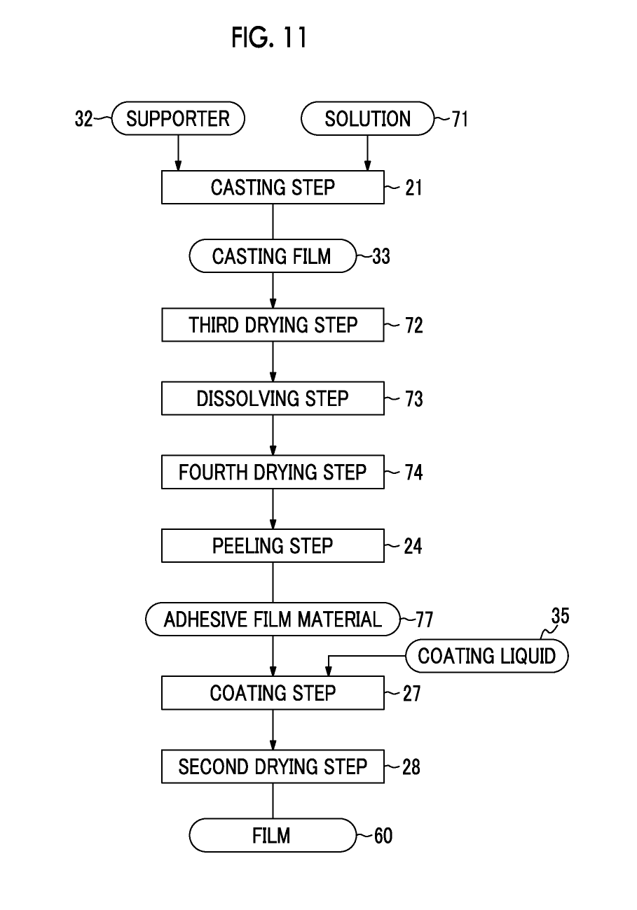

A manufacturing method of the film 60 will be described with reference to FIG. 11. The film 60 is manufactured by substituting the solution 31 of the manufacturing flow illustrated in FIG. 4 with a solution 71, and by substituting the dew condensation step 22 and the first drying step 23 with a third drying step 72, a dissolving step 73, and a fourth drying step 74.

In the solution 71, the hydrophilic polymer and the hydrophobic polymer which is the biodegradable polymer for forming the adhesive layer 61 are dissolved in a solvent. In this embodiment, the hydrophobic polymer is PLA as described above, the hydrophilic polymer is polyethylene glycol (manufactured by Wako Pure Chemical Industries, Ltd., PEG 6000), and the solvent is chloroform. The hydrophobic polymer and the hydrophilic polymer are able to be combined at an arbitrary mass ratio, and when the air gap increases, it is preferable that the mass of the hydrophilic polymer is greater than the mass of the hydrophobic polymer. In this embodiment, the mass of the hydrophobic polymer (PLA):the mass of the hydrophilic polymer (PEG) is 40:60. It is preferable that the concentration of the polymer in the solution 71 is less than or equal to 30 mass %. When the mass of the solvent (unit: g) is M3, the mass of the hydrophobic polymer (unit: g) is M4, and the mass of the hydrophilic polymer is M5 (unit: g), the concentration (unit: mass %) is a value obtained by {(M4+M5)/(M3+M4+M5)}×100. In this embodiment, the concentration is 5 mass %.

In the casting step 21, the solution 71 is dropped onto the supporter 32 and is spread on the supporter 32, and thus the casting film 33 is formed. In this embodiment, plate-like glass is used as the supporter 32, and is used at room temperature without being cooled. In the third drying step 72, dried air is supplied to the casting film 33, and thus the solvent is evaporated from the casting film 33, and the casting film 33 is dried. In the dissolving step 73, the casting film from which the solvent is evaporated is dipped in water, and the hydrophilic polymer is removed by being selectively eluted from the casting film 33. In this embodiment, the water into which the casting film is dipped is hot water at 80° C., and the dipping time is 30 minutes. In the fourth drying step 74, the casting film 33 from which the hydrophilic polymer is removed is dried by supplying dried air thereto. According to this drying, an adhesive film material 77 for forming the adhesive layer 61 is formed on the supporter 32. Thus, the adhesive film material 77 is formed of the solution 71 including the hydrophilic polymer and the hydrophobic polymer by using a water extraction method including the casting step 21, the third drying step 72, the dissolving step 73, and the fourth drying step 74. Next, the peeling step 24, the coating step 27, and the second drying step 28 are performed, and thus the film 60 is obtained.

### EXAMPLES

### Example 1 to Example 4

In Examples 1 to 3, film 10 was manufactured by using the manufacturing flow illustrated in FIG. 5. That is, the adhesive layer 11 has a honeycomb structure, and for this reason, in a section of “Structure” of “Adhesive Layer” in Table 1, “Honeycomb” is described. The average pore diameter (the average value of the diameter D) of the honeycomb structure of the adhesive layer 11 was 3 μm, and the variation coefficient of the diameter D was 3.5%. In Example 4, the film 60 was manufactured by using the manufacturing flow illustrated in FIG. 11. That is, the adhesive film material 77 was prepared by the water extraction method and the materials thereof, and the conditions described above, and the coating liquid 35 is applied onto the adhesive film material 77 and is dried, and thus the film 60 including the pores having different shapes and sizes is manufactured. In the obtained film 60, the diameter of the opening was in a range of approximately greater than or equal to 500 nm and less than or equal to 3 μm, and was uneven. For this reason, in the section of “Structure” of “Adhesive Layer” in Table 1, “Uneven Porous Structure” is described. The biodegradable polymer forming the adhesive layers 11 and 61, the material of the adhesion inhibiting layer 12, and the contact angle θb of the other film surfaces 10b and 60b are respectively described in a section of “Material” of “Adhesive Layer” in Table 1, a section of “Material” of “Adhesion Inhibiting Layer”, and a section of “Contact Angle θb” of “Adhesion Inhibiting Layer”. Furthermore, in Example 2, a polylactic acid PEG copolymer is an example of a PEG derivative.

In each of the obtained films 10 and 60, the agglutination preventive effect, the position shift in the affected area, and the repasting properties with respect to the affected area were evaluated by using the following method. The agglutination preventive effect was evaluated by performing an in vivo test using a rabbit. Specifically, the rabbit was subjected to laparotomy, each defect of interlayer peeling and peritoneum peeling was applied to the intestinal canal and the tunica serosa, each of the films 10 and 60 which was cut to have a size of 2.5 cm square was pasted to a defect portion of the intestinal canal, and then the abdominal region was sutured. The laparotomy was performed 14 days after the film was pasted, and an agglutination state of the affected area was visually confirmed. Then, the evaluation was performed on the basis of the following standard. The evaluation results are shown in a section of “Agglutination Preventive Effect” in Table 1. A and B are acceptable levels, and C is an unacceptable level.

A: The agglutination is not observed at all.

B: Weak fibrous agglutination is observed, but is allowable.

C: The intestinal canal and the peritoneum are strongly agglutinated.

In the position shift in the affected area, whether or not each of the films 10 and 60 in the affected area remained at the time of performing the laparotomy 14 days after each of the films 10 and 60 was pasted to the intestinal canal was visually confirmed. Marking was performed with respect to the affected area to which the film was pasted by using a suture thread as a marker after the pasting and before the suture of the abdominal region. Then, the evaluation was performed on the basis of the following standard. The evaluation results are shown in a section of “Position Shift in Affected Area” in Table 1. A and B are acceptable levels, and C is an unacceptable level.

A: The film remains in a marking portion.

B: The film is slightly shifted from the marking portion.

C: The film is not observed in the marking portion.

In the repasting properties with respect to the affected area, each of the films 10 and 60 which was pasted to the intestinal canal once was peeled off after leaving each of the films 10 and 60 for 10 seconds from the pasting, and the peeled films 10 and 60 were evaluated on the basis of the following standard. Then, the evaluation was performed on the basis of the following standard. The evaluation results are shown in a section of “Repasting Properties” in Table 1. A and B are acceptable levels, and C is an unacceptable level.

A: The shape of the film is maintained even after the peeling, and is able to be repasted and reused.

B: A part of the film is broken at the time of the peeling, but is able to be repasted and reused.

C: The film is not peeled off or is broken at the time of the peeling, and is not able to be reused.

TABLE 1

| | | | | | | | | Position |
|---|---|---|---|---|---|---|---|---|
| | | | Adhesion Inhibiting Layer | | | Agglutination | | Shift in |
| | Adhesive Layer | | Presence or | | Contact Angle | Preventive | Repasting | Affected |
| | Structure | Material | Absence | Material | θb (°) | Effect | Properties | Area |
| Example 1 | Honeycomb | PLA | Present | Phospholipid | 28 | A | A | A |
| Example 2 | Honeycomb | PLA | Present | Polylactic Acid | 10 | A | A | A |
| | | | | PEG Copolymer | | | | |
| Example 3 | Honeycomb | PLA | Present | Sorbitan Fatty | 15 | B | A | A |
| | | | | Acid Ester | | | | |
| Example 4 | Uneven Porous | PLA | Present | Phospholipid | 28 | A | A | B |
| | Structure | | | | | | | |
| Comparative | Flat Film | PLA | Absent | — | 80 | C | A | C |
| Example 1 | | | | | | | | |
| Comparative | Honeycomb | PLA | Absent | — | 80 | C | A | A |
| Example 2 | | | | | | | | |
| Comparative | Flat Film | PLA and | Present | Phospholipid | 28 | A | C | A |
| Example 3 | | Gelatin | | | | | | |

### Comparative Example 1

The solution 31 in which PLA is dissolved was casted onto the supporter 32, the casting film 33 was formed, the casting film 33 was dried and was peeled off from the supporter 32, and thus a film of which both surfaces were flat was prepared. For this reason, in the section of “Structure” of “Adhesive Layer” in Table 1, “Flat Film” is described. This film did not include the adhesion inhibiting layer 12, and thus “Absent” is described in a section of “Presence or Absence” of “Adhesion Inhibiting Layer” in Table 1.

In the obtained film, the agglutination preventive effect, the position shift, and the repasting properties with respect to the affected area were evaluated by using the same method and standard as those in Examples 1 to 4. The evaluation results are shown in Table 1.

### Comparative Example 2

A film not including the adhesion inhibiting layer 12 was manufactured by the casting step 21, the dew condensation step 22, the first drying step 23, and the peeling step 24 of the manufacturing flow illustrated in FIG. 5. This film was identical to the adhesive film material 34 in Example 1, and the diameter of the opening of the pore which was opened in one film surface was 3 μm. This film did not include the adhesion inhibiting layer 12, and thus “Absent” is described in the section of “Presence or Absence” of “Adhesion Inhibiting Layer” in Table 1.

In the obtained film, the agglutination preventive effect, the position shift, and the repasting properties with respect to the affected area were evaluated by using the same method and standard as those in Examples 1 to 4. The evaluation results are shown in Table 1.

### Comparative Example 3

The solution 31 in which PLA was dissolved was casted onto the supporter 32, the casting film 33 was formed, the casting film 33 was dried and was peeled off from the supporter 32, and thus film of which both surfaces were flat was prepared. Gelatin was applied onto one film surface as a gelling component. Therefore, in the section of “Material” of “Adhesive Layer” in Table 1, “PLA and Gelatin” is described. In addition, the adhesion inhibiting layer 12 was formed on the other film surface by using the same method and material as those in Example 1.

In the obtained film, the agglutination preventive effect, the position shift, and the repasting properties with respect to the affected area were evaluated by using the same method and standard as those in Examples 1 to 4. The evaluation results are shown in Table 1.

What is claimed is:

1. A film, comprising:
an adhesive layer which includes at least one opening defining an air gap which absorbs and maintains a liquid on one film surface and generates a capillary force for adhering the adhesive layer to a first cell group, and is formed of a biodegradable polymer; and
an adhesion inhibiting layer which configures the other film surface, and inhibits adhesion between a second cell group different from the first cell group and the adhesive layer,
wherein in order to separate the first cell group and the second cell group into the one film surface side and the other film surface side, the air gap is formed not to be penetrated in a thickness direction of the film.

2. The film according to claim 1,
wherein the adhesion inhibiting layer covers a surface of the adhesive layer on a side opposite to the one film surface, and thus the air gap is not penetrated in the thickness direction of the film.

3. The film according to claim 1,
wherein the adhesion inhibiting layer is formed of at least one of phospholipid, a polyethylene glycol derivative, a 2-methacryloyloxy ethyl phosphorylcholine polymer, polyoxy ethylene sorbitan fatty acid ester, polyoxy ethylene hardened castor oil, sodium dodecylsulfate, polyoxy ethylene polyoxy propylene glycol, sorbitan fatty acid ester, glycerin fatty acid ester, sucrose fatty acid ester, lecitin, saponin, and sterol.

4. The film according to claim 2,
wherein the adhesion inhibiting layer is formed of at least one of phospholipid, a polyethylene glycol derivative, a 2-methacryloyloxy ethyl phosphorylcholine polymer, polyoxy ethylene sorbitan fatty acid ester, polyoxy ethylene hardened castor oil, sodium dodecylsulfate, polyoxy ethylene polyoxy propylene glycol, sorbitan fatty acid ester, glycerin fatty acid ester, sucrose fatty acid ester, lecitin, saponin, and sterol.

5. The film according to claim 1,
wherein a contact angle of water in the other film surface is less than or equal to 40°.

6. The film according to claim 2,
wherein a contact angle of water in the other film surface is less than or equal to 40°.

7. The film according to claim 3,
wherein a contact angle of water in the other film surface is less than or equal to 40°.

8. The film according to claim 4,
wherein a contact angle of water in the other film surface is less than or equal to 40°.

9. The film according to claim 1,
wherein a diameter of the opening of the air gap on the one film surface is in a range of greater than or equal to 100 nm and less than or equal to 20 μm, a depth of the air gap is in a range of greater than or equal to 100 nm and less than or equal to 20 μm, and an aperture ratio in the one film surface is in a range of greater than or equal to 20% and less than or equal to 80%.

10. The film according to claim 2,
wherein a diameter of the opening of the air gap on the one film surface is in a range of greater than or equal to 100 nm and less than or equal to 20 μm, a depth of the air gap is in a range of greater than or equal to 100 nm and less than or equal to 20 μM, and an aperture ratio in the one film surface is in a range of greater than or equal to 20% and less than or equal to 80%.

11. The film according to claim 3,
wherein a diameter of the opening of the air gap on the one film surface is in a range of greater than or equal to 100 nm and less than or equal to 20 μm, a depth of the air gap is in a range of greater than or equal to 100 nm and less than or equal to 20 μm, and an aperture ratio in the one film surface is in a range of greater than or equal to 20% and less than or equal to 80%.

12. The film according to claim 4,
wherein a diameter of the opening of the air gap on the one film surface is in a range of greater than or equal to 100 nm and less than or equal to 20 μm, a depth of the air gap is in a range of greater than or equal to 100 nm and less than or equal to 20 μm, and an aperture ratio in the one film surface is in a range of greater than or equal to 20% and less than or equal to 80%.

13. The film according to claim 5,
wherein a diameter of the opening of the air gap on the one film surface is in a range of greater than or equal to 100 nm and less than or equal to 20 μm, a depth of the air gap is in a range of greater than or equal to 100 nm and less than or equal to 20 μm, and an aperture ratio in the one film surface is in a range of greater than or equal to 20% and less than or equal to 80%.

14. The film according to claim 6,
wherein a diameter of the opening of the air gap on the one film surface is in a range of greater than or equal to 100 nm and less than or equal to 20 μm, a depth of the air gap is in a range of greater than or equal to 100 nm and less than or equal to 20 μm, and an aperture ratio in the one film surface is in a range of greater than or equal to 20% and less than or equal to 80%.

15. The film according to claim 7,
wherein a diameter of the opening of the air gap on the one film surface is in a range of greater than or equal to 100 nm and less than or equal to 20 μm, a depth of the air gap is in a range of greater than or equal to 100 nm and less than or equal to 20 μm, and an aperture ratio in the one film surface is in a range of greater than or equal to 20% and less than or equal to 80%.

16. The film according to claim 8,
wherein a diameter of the opening of the air gap on the one film surface is in a range of greater than or equal to 100 nm and less than or equal to 20 μm, a depth of the air gap is in a range of greater than or equal to 100 nm and less than or equal to 20 μm, and an aperture ratio in the one film surface is in a range of greater than or equal to 20% and less than or equal to 80%.

17. The film according to claim 9,
wherein the adhesive layer has a honeycomb structure due to the opening of the air gap in the one film surface.

18. The film according to claim 10,
wherein the adhesive layer has a honeycomb structure due to the opening of the air gap in the one film surface.

19. The film according to claim 17,
wherein the adhesive layer is formed by causing dew condensation on a film formed of a solution in which a hydrophobic biodegradable polymer is dissolved in a hydrophobic solvent, and by evaporating the solvent and a water droplet generated due to the dew condensation.

20. The film according to claim 18,
wherein the adhesive layer is formed by causing dew condensation on a film formed of a solution in which a hydrophobic biodegradable polymer is dissolved in a hydrophobic solvent, and by evaporating the solvent and a water droplet generated due to the dew condensation.
